(19) United States
(12) Patent Application Publication
(10) Pub. No.: US 2003/0077839 A1
(43) Pub. Date: Apr. 24, 2003

(54) Method and apparatus for recovering biomolecules

(76) Inventor: Hiroyuki Takei, Hatoyama (JP)

Correspondence Address: REED SMITH LLP, Suite 1400, 3110 Fairview Park Drive, Falls Church, VA, 22042, US

(21) Appl. No.: 10/210,087

(22) Filed: Aug. 2, 2002

(30) Foreign Application Priority Data
Oct. 18, 2001 (JP) 2001-320061

(51) Int. Cl. G01N 33/53; G01N 1/18

(52) U.S. Cl. 436/177; 436/178; 436/164; 436/165; 436/172; 435/7.1; 422/82.05; 422/82.08; 422/82.09; 422/101

(57) ABSTRACT

To detect interactions of biomolecules with high sensitivity and to efficiently recover the biomolecules, a biomolecule or cell is attached to a surface of a fine particle immobilized on a substrate, and the biomolecules or cells attached to the fine particle is recovered. This configuration provides a high recovery rate after checking the presence of the biomolecules.

### FIELD OF THE INVENTION

The present invention relates to methods and apparatus for detecting interactions of biomolecules and biotissues with high sensitivity and for efficiently recovering these biomolecules and biotissues. Such biomolecules and biotissues include, for example, proteins, nucleic acids, antibodies, hormones, and cells.

### BACKGROUND OF THE INVENTION

JP-A-326193/1999 discloses an apparatus for adsorbing and detecting biomolecules, utilizing optical properties of gold fine particles formed on a surface of a substrate. The apparatus selectively captures a biomolecule in a liquid on a solid-phase surface and detects the same without labeling. With reference to FIG. 11A, the surface of a sensor constituting the solid-phase surface has a microstructure, in which a substrate 152 is coated with a gold thin film 151 having a thickness of 20 nm. The substrate 152 carries at least one polymeric or inorganic fine particle 154 having a particle diameter of 100 nm. The polymeric fine particle 154 is coated with a film of gold 153 having a thickness of 20 nm. With reference to FIG. 11B, this structure has noticeable absorption characteristics, and its maximal absorption wavelength varies according to a refraction index in the vicinity of the fine particle. Accordingly, when the surface of the fine particle is modified with, for example, an antibody 157 selectively binds an antigen 158 specific to the antibody 157, the maximal absorption wavelength starts to vary at the time 159 when the antibody begins to bind the antigen. Specifically, a peak wavelength 155 in absorbance shifts to a peak wavelength 156, and how the adsorption occurs and proceeds can be monitored by determining the maximal absorption wavelength.

Alternatively, such biomolecules are conventionally purified by a process using a column chromatography technique (“Protein Purification-Principles and Practice”, Robert K. Scopes, Springer-Verlag Tokyo).

According to the ion exchange chromatography technique, an ion exchanger comprising diethylaminoethyl cellulose or carboxymethyl cellulose statistically attach a target biomolecule on its surface, and other substances than the target biomolecule are washed away. A buffer having a different pH or salt concentration is then passed through the column so as to elute and recover the biomolecule. By using an immunoadsorbent carrying an immobilized antibody as the column, the biomolecule can be attached to the column surface with higher selectivity. Thereafter, a buffer having a different pH or salt concentration is passed through the column so as to elute and recover the biomolecule.

U.S. Pat. No. 6,093,370 discloses a process in which a DNA fragment is hybridized on a DNA chip and is then separated from the substrate by local heating induced by laser irradiation.

Alternatively, such a biomolecule is recovered by the use of a surface plasmon resonance sensor (“Real-Time Analysis of Biomolecular Interactions-Applications of BIACORE”, Kazuhiro NAGATA and Hiroshi HANDA, Springer-Verlag Tokyo). The principle of this process is shown in FIG. 2. A surface plasmon resonance sensor includes a prism 22 coated with gold 21. When a surface of the prism 22 is modified electrostatically or with an antibody 23, a biomolecule 25 is introduced via a fine channel 24 formed on the prism surface and is then captured or trapped by the modified prism surface. The reflection coefficient of monochromatic light 26 applied from the bottom of the prism 22 varies according to an incident angle and the refractive index on the prism surface. The relationship between the reflection coefficient and the incident angle is shown as a sensorgram 27. Upon the change of the refractive index of the surface due to the attachment of the biomolecules, the sensorgram 27 changes to a sensorgram 28. The attachment of the biomolecules can therefore be monitored by observing the change in sensorgram. After checking that the attachment occurs, a buffer having a different pH or salt concentration is passed via a channel to elute the biomolecules. By constituting the fine channel 24 with, for example, a pump 30 and a valve 31 controlled by a computer 29, the target biomolecules can be recovered into any desired biomolecule recovery chamber 32.

According to the aforementioned conventional processes using a small-capacity column or a designed fine channel on a prism, biomolecules can be recovered to some extent from a specimen in a small amount.

However, some of the eluted biomolecules are inevitably lost due to physical attachment to the surface of the fine channel. For example, when a specimen on the order of about 100 fm is supplied to a mass spectrometer in a biomolecule interaction sensor, in which the specimen is introduced via a fine channel, the specimen is lost by half one hour later. When the recovered specimen is aspirated into an injection needle, the quantity is further reduced (“Real-Time Analysis of Biomolecular Interactions-Applications of BIACORE”, Kazuhiro NAGATA and Hiroshi HANDA, Springer-Verlag Tokyo).

### SUMMARY OF THE INVENTION

An object of the present invention is to efficiently recover such biomolecules.

Another object of the present invention is to recycle/reuse the particles for recovering biomolecules.

According to one aspect of the invention, the method for recovering biomolecules includes the steps of: bringing a specimen containing target biomolecules into contact with at least one fine particle being formed on a substrate for carrying at least one probe so as to bind the probe with the target biomolecules; monitoring the biomolecules on the substrate by using an optical property of the fine particle without labeling the biomolecules; separating and collecting the fine particle from the substrate by a physical property of the fine particle; and recovering the biomolecules from the fine particle.

According to another aspect of the invention, the apparatus for recovering biomolecules includes: a substrate carrying at least one fine particle on its surface, and the fine particle carries at least one probe; an inlet for importing a specimen containing the biomolecules capable of being bound with the probe; a measuring instrument for determining attachment of the biomolecules to the substrate by the probe; a monitoring means for monitoring the biomolecules attached to the substrate using an optical property of the fine particle without labeling the biomolecules; a separation unit for separating the fine particle from the substrate; and a recovery unit for recovering the fine particle.

According to a third aspect of the invention, the method for recovering biomolecules, includes the steps of: bringing a specimen containing target biomolecules into contact with at least one noble metal-dielectric composite fine particle being formed on a substrate for carrying at least one probe so as to bind the biomolecules with the probe; separating and collecting the fine particle from the substrate by a physical property of the fine particle; and recovering the biomolecules from the find particle.

### BRIEF DESCRIPTION OF THE DRAWINGS

The foregoing and additional features and characteristics of the present invention will become more apparent from the following detailed description considered with reference to the accompanying drawings in which like reference numerals designate like elements and wherein:

FIGS. 1A, 1B and 1C are diagrams each schematically illustrating the essence of the present invention;

FIG. 2 shows the configuration and principle of a conventional apparatus;

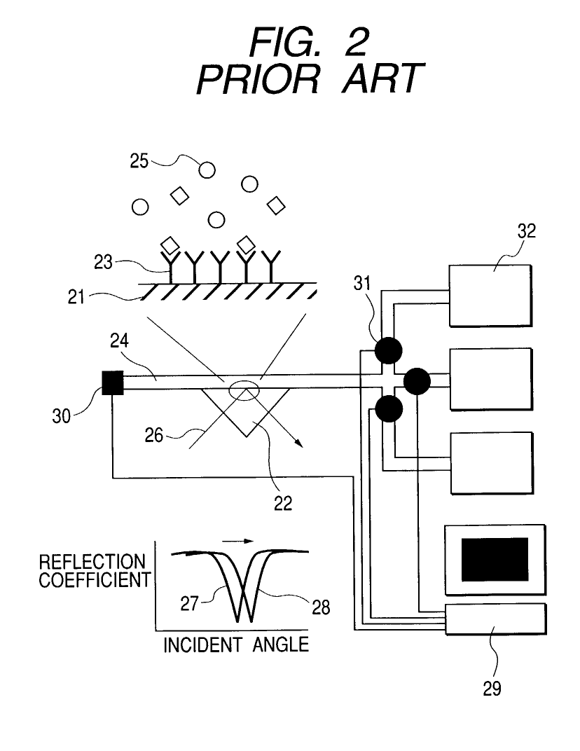

FIGS. 3A, 3B and 3C are diagrams each showing a process for monitoring whether a biomolecule is attached based on the optical properties of fine gold particles;

FIGS. 4A, 4B, 4C and 4D are diagrams each showing a process for separating the fine particle from a substrate;

FIGS. 5A, 5B, 5C and 5D are diagrams each showing a process for recovering the separated fine particles;

FIGS. 6A, 6B, 6C and 6D are diagrams showing an example in which the fine particle separated by ultrasonic irradiation is recovered using a filter;

FIGS. 7A, 7B and 7C are diagrams showing another example in which the fine particle mechanically separated is recovered by centrifugal separation;

FIGS. 8A and 8B are diagrams showing another example in which the fine particle separated by laser irradiation is recovered by using a magnet;

FIG. 9 shows another example in which plural types of target biomolecules are recovered;

FIG. 10 shows another example in which a fine particle immobilized on a disc-shaped substrate is separated by laser irradiation and is then recovered by a centrifugal force;

FIGS. 11A and 11B show the configuration and principle of a conventional apparatus;

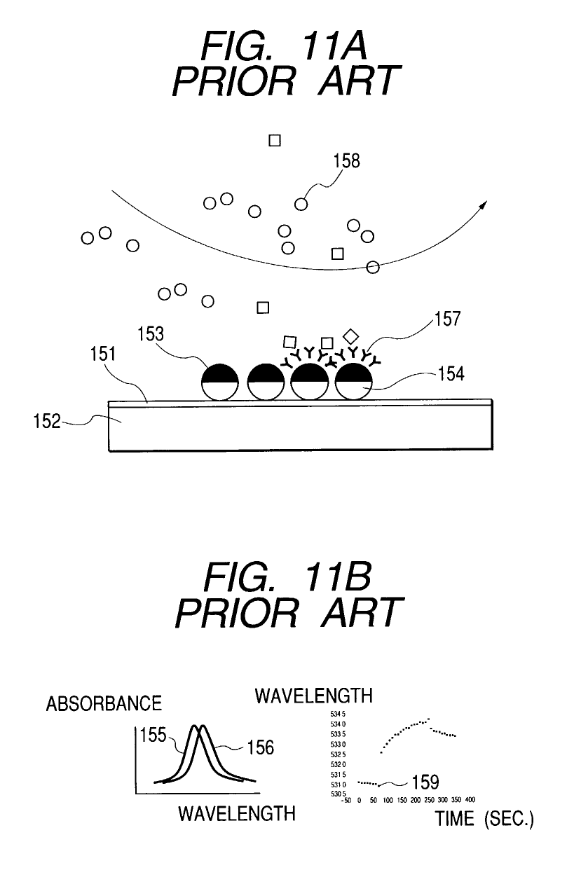

FIGS. 12 is a diagram showing how the fine particle is separated from the substrate;

FIGS. 13A, 13B and 13C are diagrams each showing a process for determining the concentration of the separated fine particle;

FIGS. 14A and 14B are diagrams each showing a process in which a buffer is replaced with a new buffer; and

FIG. 15 is a diagram showing an apparatus for automatic purifying and concentrating of a specimen.

### DETAILED DESCRIPTION OF THE PREFERRED EMBODIMENTS

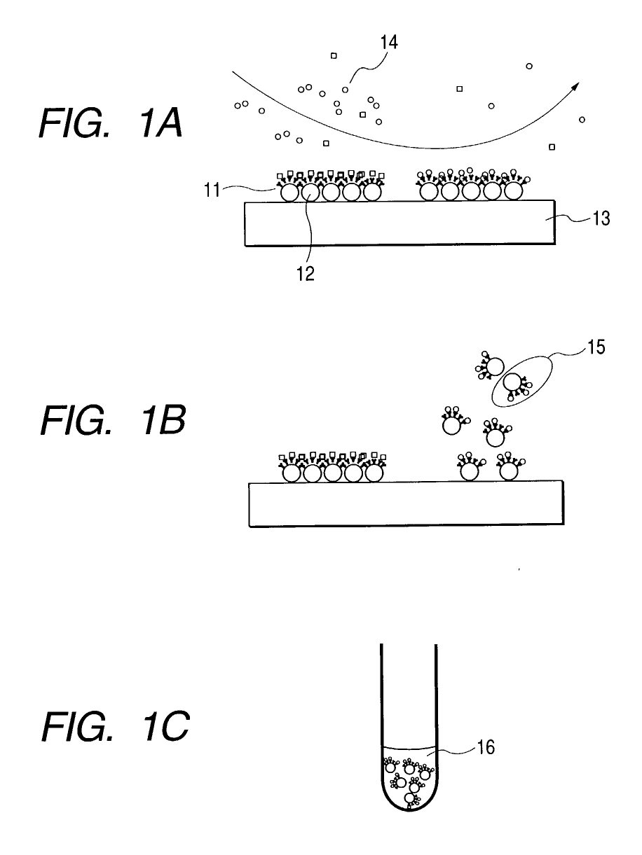

FIGS. 1A, 1B and 1C illustrate a method according to the present invention to achieve the above object. In this method, a fine particle 12 is immunologically modified on its surface electrostatically or with an antibody 11 and is formed on a substrate 13 so as to capture a biomolecule 14 (FIG. 1A). After adsorbing the biomolecule on its surface, the fine particle itself is separated from the substrate (FIG. 1B) and then the target biomolecule is recovered with the fine particle by the use of physical properties of the fine particle without elution (FIG. 1C). Such physical properties of the fine particle include magnetic properties electrostatic properties, and specific gravity. Detailed procedures to recover the biomolecules by the use of the physical properties of the fine particle will be described later. A wide variety of recovering procedures can be used in this method in contrast to the conventional recovering processes based on the physical properties of the target biomolecules.

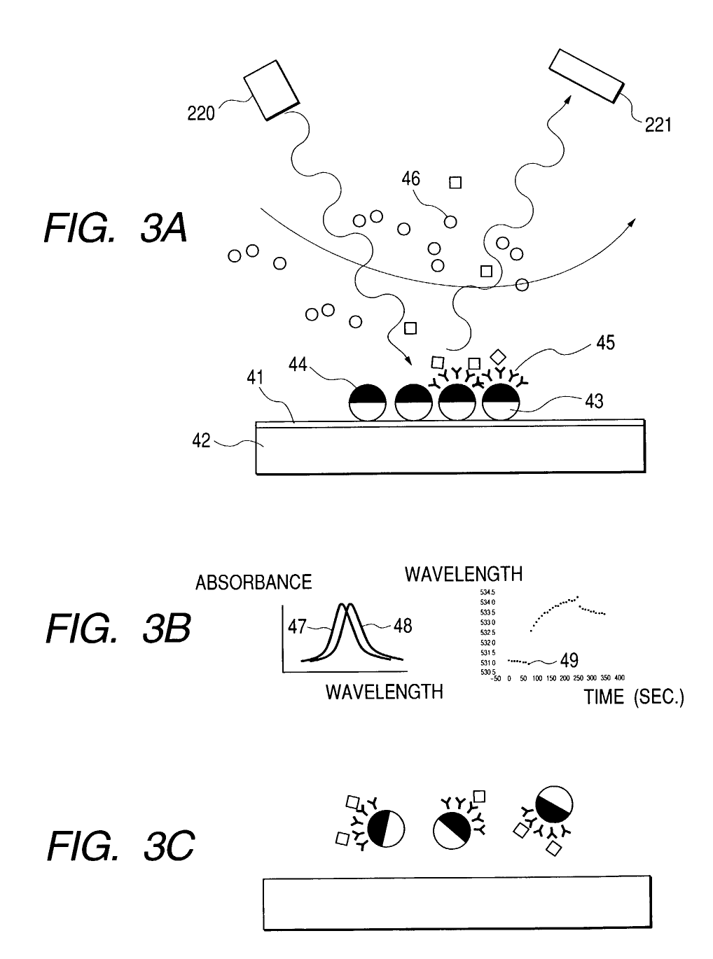

Such biomolecules attached to the surface of the fine particle can be monitored by using the optical properties of a noble metal-dielectric composite fine particle (FIGS. 3A, 3B and 3C) without labeling the biomolecules. However, the traditional labeling (with such as fluorescence, radio isotope) can be applied in conjunction with the present invention. The noble metal-dielectric composite fine particle can be prepared by a method invented by the present inventors. The noble metal-dielectric composite fine particle changes its color upon the attachment of the biomolecules on its surface. The noble metal-dielectric composite fine particle is formed on a substrate. The term “noble metal-dielectric composite fine particle” as used herein includes (but not limit to) a dielectric fine particle with a particle diameter of 10 nm to 100 μm that is formed on a substrate and carries a layer of a noble metal having a thickness of 2 nm to 40 nm. Initially, a gold thin film 41 having a thickness of 10 nm to 40 nm is formed on a substrate 42 by vapor deposition, and a layer of dielectric fine particle 43 having a particle size of 30 nm to 300 nm is formed thereon. Subsequently, a film of a noble metal such as gold is formed with a thickness of 10 nm to 40 nm by vapor deposition so as to yield the dielectric fine particle 43 carrying a noble metal fine particle 44 as a cap which is optional. Thus, the fine particle 43 carrying the noble metal layer on its upper surface is formed. The surface of the noble-metal fine particle 44 is modified with at least one probe biomolecule 45. Such probe biomolecules include, for example, DNAs, antibodies, receptors, or enzymes. A specimen containing a target biomolecule 46 is then supplied to thereby form a specific binding. As a result, the reflection spectrum 47 of the whole substrate changes to a reflection spectrum 48 so as to optically monitor the presence of the target biomolecules 46. FIG. 3B shows that the maximal absorption wavelength of a reflection spectrum varies from the time point when the attachment begins. The change in maximal absorption wavelength can be detected according to the method described in JP-A-326193/1999. Specifically, the substrate 42 is irradiated with light from a light source 220, and the resulting reflected light is detected by a detector 221. This method described in the prior art reference is directed to detect interactions of biomolecules but not to recover such biomolecules. The binding between the dielectric fine particle and the substrate is set relatively weak such that the biomolecules on the noble metal-dielectric composite fine particle can be separated from the substrate (FIG. 3C). Detailed procedures to separate the fine particle with the biomolecules will be illustrated later. Nearly 100% of the noble metal-dielectric composite fine particles can be recovered by utilizing its physical properties, such as magnetic properties, electrostatic properties, or specific gravity. Recovering procedures will also be illustrated later. According to this method, the recovery of the biomolecules is the same with that of the noble metal-dielectric composite fine particles as long as one noble metal-dielectric composite fine particle is attached with the biomolecules on its surface. Accordingly, the recovery of the biomolecules can continuously be checked by monitoring the amount of the noble metal-dielectric composite fine particles, which amount can be easily determined. Procedures for determining the amount of the fine particles will be illustrated later. In contrast, a biomolecule eluted into a liquid phase according to conventional processes is attached to a solid-phase surface, and their attachment to an analyzing means cannot significantly be prevented or controlled. In addition, the amount of an ultimately recovered specimen cannot easily be determined. Specifically, the conventional processes recover the target biomolecules with a recovery rate of 50% or less, and an actual recovery cannot significantly be indicated in terms of numerical values. In contrast, according to the method of the present invention, the target biomolecules attaching to the noble metal-dielectric composite fine particle on its surface can be recovered with a recovery rate of 90% or more, and an actual recovery can easily be determined. Procedures for determining the recovery rate will be illustrated later.

As is described above, the present invention has, in one aspect, the following configuration. Initially, a polymeric or inorganic dielectric fine particle covered with a noble metal is formed on a substrate. A specific binding of biomolecules on the surface of the noble metal is optically detected. The noble metal-dielectric composite fine particle is then separated from the substrate so as to recover and concentrate the biomolecules. Such polymers for use herein include, for example, polystyrenes, dextran, styrene-butadiene copolymers, polyvinyltoluenes, styrene-divinylbenzene copolymers, or vinyltoluene-t-butylstyrene copolymers. Inorganic materials for use herein include, for example, silicon, silicon oxides, or titanium oxides.

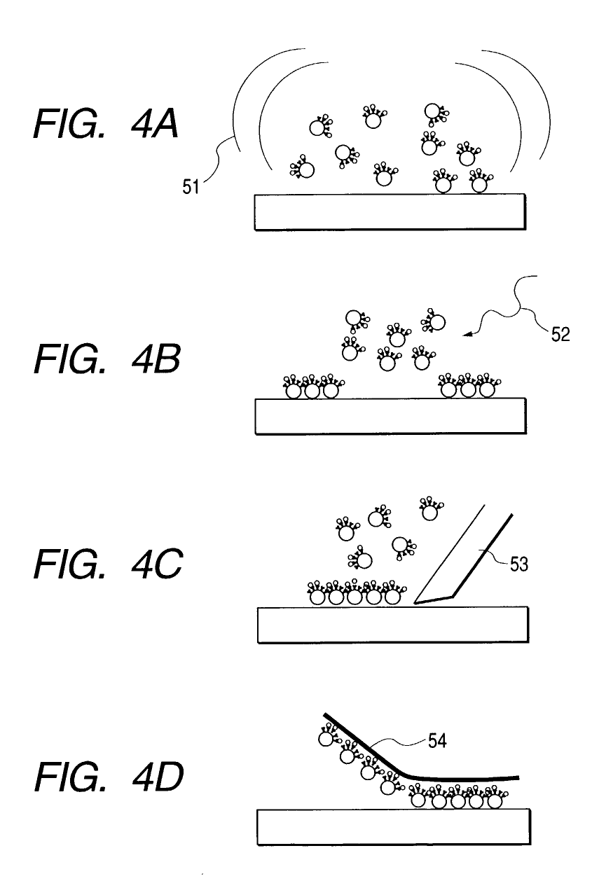

Next, the noble metal-dielectric composite fine particle may be separated from the substrate in the following manner. Specifically, the noble metal-dielectric composite fine particle is separated from the substrate by ultrasonic irradiation 51 (FIG. 4A), by laser irradiation 52 (FIG. 4B), by mechanical separating with the use of a scraper 53 (FIG. 4C), or by the use of a pressure-sensitive adhesive tape 54 (FIG. 4D). For example, upon separation by ultrasonic irradiation, an ultrasonic wave 76 is applied to the substrate in a buffer at an oscillation frequency of 10 kHz to 100 kHz and a power of 10 W to 1000 W for 1 millisecond to 100 seconds. Upon separation by laser irradiation, 1 to 100 pulses of a fundamental, double harmonic or triple harmonic wave of YAG laser at an intensity of 1 mJ/cm² to 50 mJ/cm² or 1 to 100 pulses of nitrogen laser light 131 at an intensity of 1 mJ/cm² to 50 mJ/cm² is applied to the noble metal-dielectric composite fine particle in a buffer or in the air. Alternatively, 1 to 100 pulses of excimer laser light 144 at an intensity of from 1 mJ/cm² to 50 mJ/cm² is applied to the noble metal-dielectric composite fine particle in a buffer or in the air. Such irradiation of pulsed laser light can avoid damages to the biomolecules. As the scraper, those having a surface made of rubber, plastics, paper or wood are preferred. The noble metal-dielectric composite fine particle can be separated from the substrate using the scraper either in a buffer or in the air. Pressure-sensitive adhesive tapes generally used as stationery can be used as the pressure-sensitive adhesive tape of the invention. Such pressure-sensitive adhesive tapes are preferably capable of applying force of 100 N/cm² or more when they are applied onto a flat surface of gold. When the pressure-sensitive adhesive tape is used, the noble metal-dielectric composite fine particle is separated from the substrate in the air. The noble metal-dielectric composite fine particle may be separated from the substrate mechanically or by the application of laser light in a buffer or in the air, as described above. When the probe and the target biomolecules attached to the probe are substances resistant to dryness, the noble metal-dielectric composite fine particle is separated in the air. When the probe and the biomolecules are susceptible to dryness, the noble metal-dielectric composite fine particle is separated in a buffer.

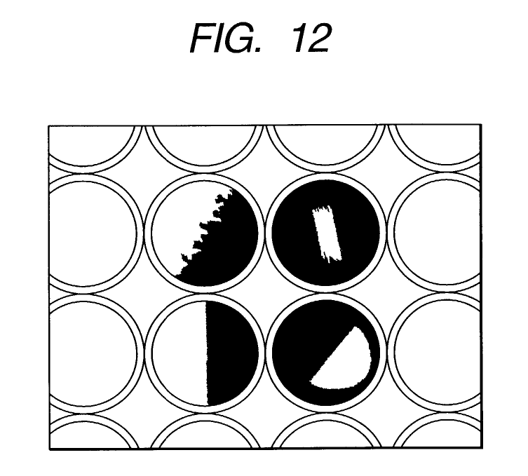

According to any of the aforementioned procedures, the noble metal-dielectric composite fine particle can be separated from the substrate with an efficiency of equal to or more than 95%. FIG. 12 shows a sample in which the noble metal-dielectric composite fine particle is separated by ultrasonic irradiation (top left); a sample in which the noble metal-dielectric composite fine particle is separated by laser irradiation and the gold thin film of the substrate is exposed (bottom left); a sample in which the noble metal-dielectric composite fine particle is separated using a scraper and the gold thin film of the substrate is exposed (top right); and a sample in which the noble metal-dielectric composite fine particle is separated using a pressure-sensitive adhesive tape and the gold thin film of the substrate is exposed (bottom right). This figure also shows a color developed due to the absorption of the biomolecules by the noble metal-dielectric composite fine particle lightens the reflection by the gold thin film on the substrate significantly and increases with the separation of the noble metal-dielectric composite fine particle from the substrate by ultrasonic irradiation, by laser irradiation, by the use of a scraper, or by the use of a pressure-sensitive adhesive tape, respectively.

The advantages of each of the separating procedures will now be described. By ultrasonic irradiation, the noble metal-dielectric composite fine particle can be separated uniformly from the overall substrate under mild irradiation conditions that do not damage proteins and other high-molecular weight biomolecules. By laser irradiation, the noble metal-dielectric composite fine particle can selectively be separated from any optional region on the substrate so as to easily recover a desired type of target biomolecules when plural types of biomolecules are adsorbed in different regions. By using a scraper, the noble metal-dielectric composite fine particle can most easily be separated from the substrate. By using a pressure-sensitive adhesive tape, plural types of biomolecules can be recovered and stored while keeping relative locations of individual biomolecules when they are attached in different regions.

The noble metal-dielectric composite fine particle immediately after separation is dispersed in a buffer or is in the air.

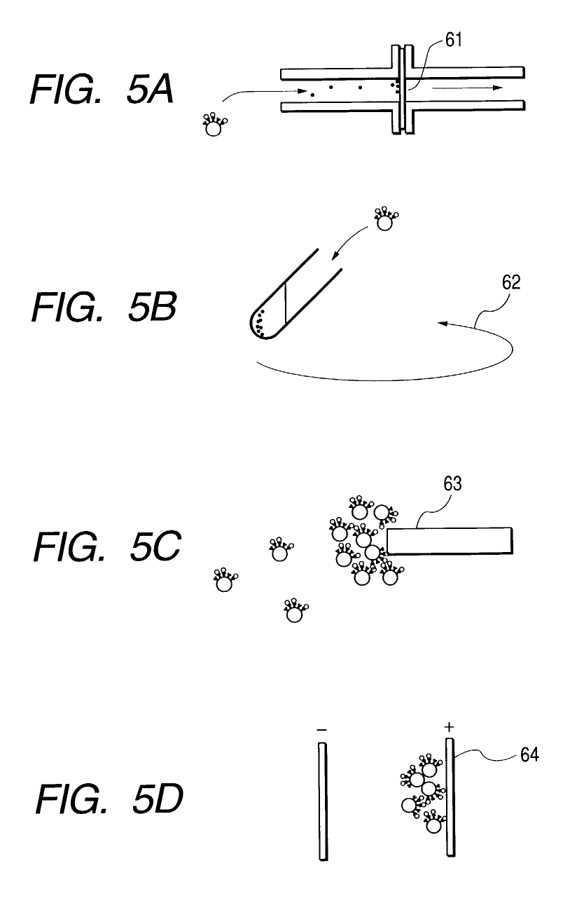

Next, the separated noble metal-dielectric composite fine particle can be recovered according to the following procedures. After undergoing the separating step, the noble metal-dielectric composite fine particle is dispersed in a buffer or is in the air. When the noble metal-dielectric composite fine particle is in the air, initially, it should be dispersed in a buffer in the recovering step. Specifically, when the noble metal-dielectric composite fine particle is separated in the air mechanically or by laser irradiation, the separated noble metal-dielectric composite fine particle is then placed in a buffer. When the noble metal-dielectric composite fine particle is separated by the use of a pressure-sensitive adhesive tape, the pressure-sensitive adhesive tape carrying the noble metal-dielectric composite fine particle is immersed in a liquid so as to separate the noble metal-dielectric composite fine particle from the pressure-sensitive adhesive tape. Alternatively, the pressure-sensitive adhesive tape is dissolved in a solvent to separate the noble metal-dielectric composite fine particle from the pressure-sensitive adhesive tape. Thus, a buffer containing the dispersed noble metal-dielectric composite fine particle is provided, then the noble metal-dielectric composite fine particle is recovered from the buffer. It can be recovered by the use of a filter 61 (FIG. 5A), a centrifugal separator 62 (FIG. 5B), a noble metal-dielectric composite fine particle containing a super paramagnetic substance as the noble metal-dielectric composite fine particle and recovering the noble metal-dielectric composite fine particle by the use of a magnet 63 (FIG. 5C), or a noble metal-dielectric composite fine particle carrying an electric charge as the noble metal-dielectric composite fine particle and electrostatically recovering the noble metal-dielectric composite fine particle using an electrode 64 (FIG. 5D). When the noble metal-dielectric composite fine particle is recovered by the use of a filter, the filter preferably has a pore size about four fifth of the diameter of the noble metal-dielectric composite fine particle. Upon the recovery with the centrifugal separator 62, the noble metal-dielectric composite fine particle is subjected to centrifugation at a centrifugal force of equal to or more than 150000 g. Upon the recovery with a magnet, the magnet preferably generates a magnetic field with a surface inductive flux of 1000 gausses or more. Upon the electrostatic recovery, an electrode made of platinum or gold may be used at a voltage of 0.1 to 2 V.

The recovery efficiencies of using the filter, centrifugal separator, magnet and electrode as above are equal to or more than 95%, equal to or more than 90%, equal to or more than 90% and equal to or more than 90%, respectively.

The advantages of each of the recovery procedures will now be described. By recovering with a filter, the noble metal-dielectric composite fine particle can be rapidly recovered in a short time of several seconds or less. The use of a centrifugal separator can easily recover the target substance. The use of a magnet can rapidly recover the target substance with satisfactory control. The use of an electrode can miniaturize the recovery system.

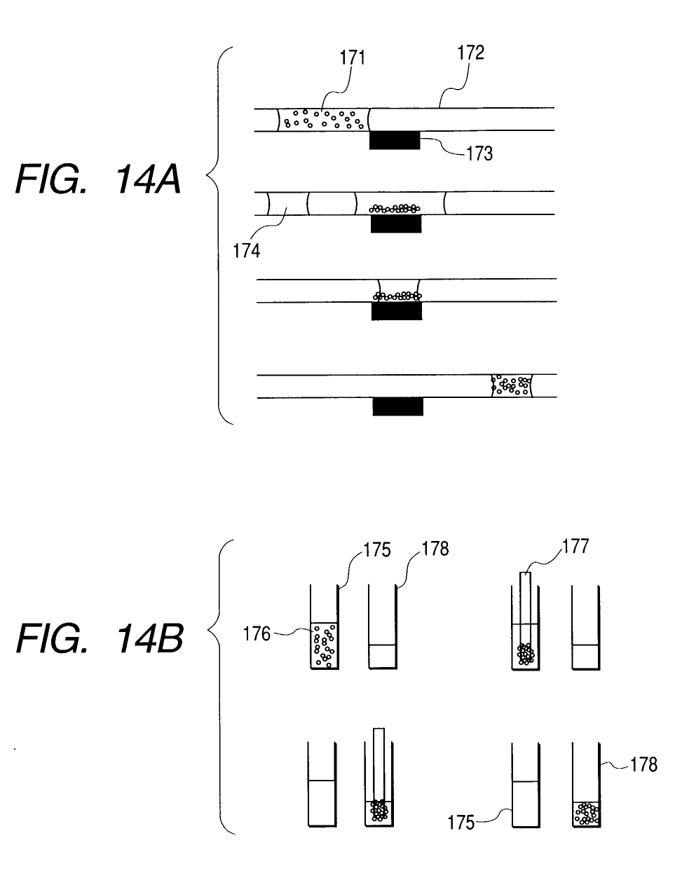

Another procedure for recovering the noble metal-dielectric composite fine particle will be illustrated below. When the target biomolecules are ultimately dissolved into a liquid phase, its concentration varies according to the volume of a solution containing the noble metal-dielectric composite fine particles. It is easy to recover the noble metal-dielectric composite fine particles from a liquid and to re-dissolve the same in another liquid. The biomolecules can substantially be concentrated by re-dissolving the noble metal-dielectric composite fine particle in a small amount of a liquid and thereby eluting the biomolecules. FIGS. 14A and 14B each show a recovery procedure of the noble metal-dielectric composite fine particle. FIG. 14A shows a process in which the noble metal-dielectric composite fine particle is temporarily and locally held, whereas liquids are moved. In this process, a super paramagnetic noble metal-dielectric composite fine particle is used. A suspension 171 containing the super paramagnetic noble metal-dielectric composite fine particle is passed through a fine channel 172 so as to capture or trap with an electromagnet 173 the noble metal-dielectric composite fine particle. The liquid alone is moved away from the region of the electromagnet 173, and the super paramagnetic noble metal-dielectric composite fine particle is then re-dissolved in a less amount of another liquid 174. Alternatively, the noble metal-dielectric composite fine particle may be collected and moved. Specifically, the super paramagnetic noble metal-dielectric composite fine particle in a chamber 175 is collected using an electromagnet 177 which is then moved to another chamber 178, and the super paramagnetic noble metal-dielectric composite fine particle is re-dissolved in a liquid in the chamber 178. The chamber 178 contains a less amount of the liquid than that in the chamber 175. The specimen as a concentrate can be reused by moving the noble metal-dielectric composite fine particle to a region where the specimen is required according to the object of the recovery and eluting the biomolecules at a minimal amount of a liquid. To elute the biomolecules, 10 mM glycine-hydrochloric acid buffer of pH 3.0 or lower, a high concentration sodium chloride aqueous solution of 1 M or more, 8M guanidine hydrochloride as a protein denaturing agent, or 50% ethylene glycol or other solvents are used. The elution procedure can be selected depending on the type of the target biomolecules. In contrast, upon elution of a specimen according to the conventional processes, a small amount of a liquid after elution is difficult to handle and the specimen is inevitably lost due to their attachment to a solid-phase surface of an analyzing means.

The noble metal-dielectric composite fine particle is recovered from the buffer according to the above procedures.

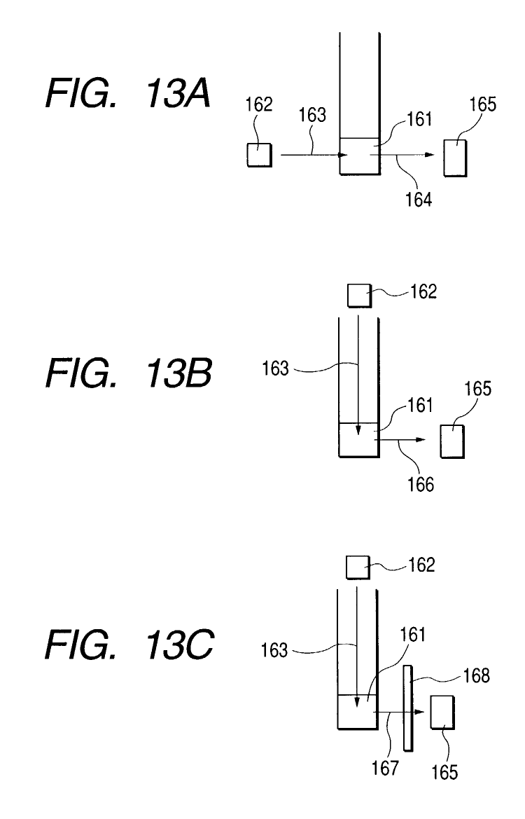

Next, the recovery of the noble metal-dielectric composite fine particle can be determined according to the following procedures. The recovery is determined by obtaining the recovery data, and this step is not always performed in the recovery of the noble metal-dielectric composite fine particle. Such determination processes of the recovery include a process in which the recovery is determined based on the light absorption of the noble metal-dielectric composite fine particle (FIG. 13A), a process in which the recovery is determined based on light scattering by the noble metal-dielectric composite fine particle (FIG. 13B), and a process in which a noble metal-dielectric composite fine particle containing a fluorescent dye such that the recovery is determined based on fluorescent signal strength (FIG. 13C). Upon determination, near-infrared radiation is preferably applied, since the noble metal-dielectric composite fine particle itself exhibits significant absorption and scattering in near-infrared wavelength regions. In FIGS. 13A, 13B and 13C, the reference numerals are as follows: 161: a suspension of the noble metal-dielectric composite fine particle; 162: a light source; 163: an irradiated light; 164: a transmitted light; 165: a photodetector; 166: a scattered light; 167: a fluorescent light; and 168: a cut-off filter.

Different advantages are provided by each of the determination processes of the recovery. The determination based on light absorption or scattering can easily determine the recovery. The use of a fluorescent dye can determine the recovery with high sensitivity.

### EXAMPLE 1

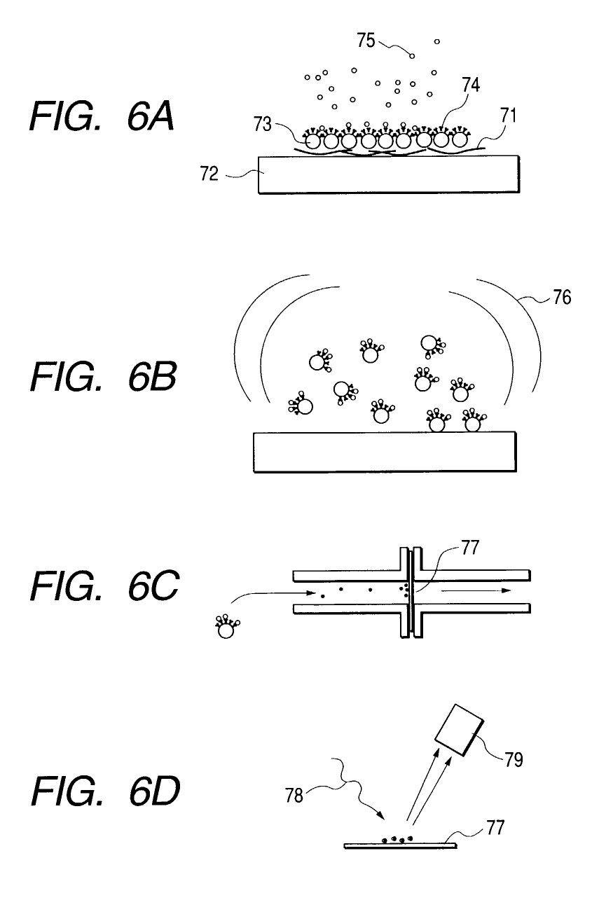

FIGS. 6A, 6B, 6C and 6D show an example according to the present invention. A silicon substrate 72 coated with a polylysine 71 is allowed to electrostatically adsorb on its surface a polystyrene fine particle 73 having a particle diameter of 30 nm to 100 μm, and the polystyrene then physically adsorbs an antibody 74 on its surface. A specimen containing a kind of target biomolecule antigen 75 flows onto the substrate so as to be captured on the surface of the fine particle 73. Next, irradiation of the substrate with an ultrasonic wave 76 in water at an oscillation frequency of 50 kHz and an output of 10 W for 5 seconds separates the polystyrene fine particle 73 form the silicon substrate 72. The polystyrene fine particle 73 is then recovered using a filter 77 and is eluted with 10 mM glycine-hydrochloric acid buffer (pH 3), and the eluted antigen 75 is analyzed by laser irradiation using, for example, a mass spectrometer 79.

### EXAMPLE 2

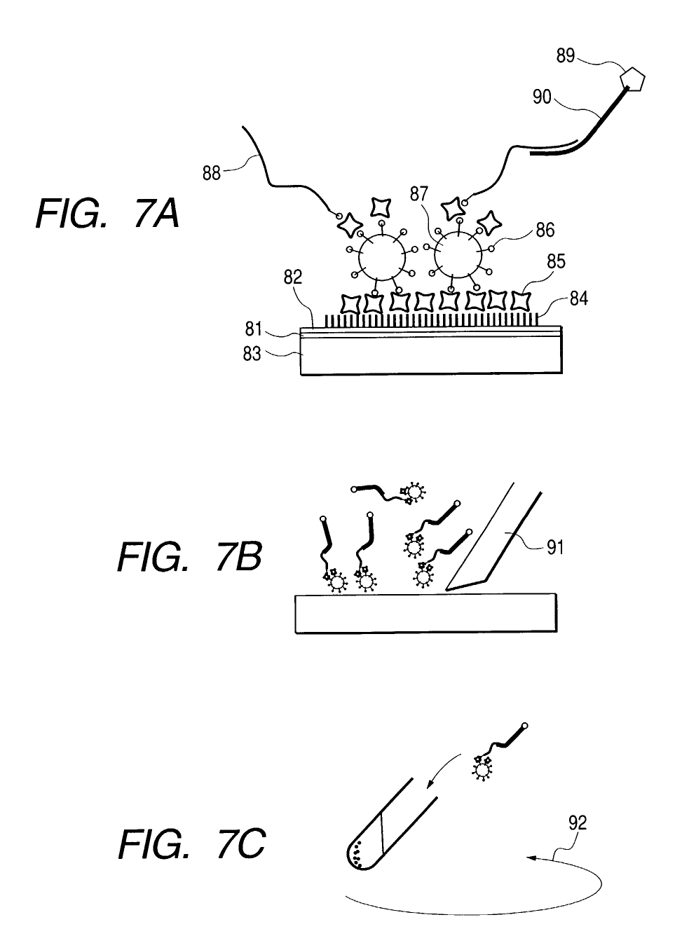

FIGS. 7A, 7B and 7C show another example according to the present invention. A glass substrate 83 carrying a chromium thin film 81 with a thickness of 2 nm and a gold thin film 82 with a thickness of 50 nm is prepared. An alkanethiol containing from 3 to 20 carbon atoms and an amino group is suspended in ethanol in a concentration ranging from 1 μM to 10 mM. The glass substrate 83 is immersed in the alkanethiol solution a day to form an alkanethiol monomolecular layer 84 on the surface of the gold thin film 82. Next, avidin protein 85 suspended in a carbodiimide solution is then added thereto, and the avidin protein 86 is immobilized on the substrate due to a condensation reaction between the carboxyl group of the surface of the avidin protein 85 and the amino group of the alkanethiol 84. A polystyrene fine particle 87 with a diameter of 30 nm to 100 μm and being coated with biotin molecules 86 is added thereto, and the polystyrene fine particle 87 is then immobilized on the substrate due to a specific binding therebetween. An avidin protein solution in a concentration of from 1 μg/ml to 10 mg/ml is then added thereto so as to bind the avidin protein to the biotin 86 on the polystyrene fine particle 87, followed by addition of biotinylated DNA 88 to thereby immobilize the biotinylated DNA 88 onto the polystyrene fine particle 87. Thereafter, a specimen containing a DNA 90 having a complementary sequence labeled with a fluorescent dye 89 flows onto the substrate and is then captured by hybridization. This device can be used for diagnosis as a DNA chip. After checking with the fluorescent dye of the DNA to ensure that the DNA is bound with the fine particle, the fine particle is mechanically separated from the substrate 83 using a scraper 91 and is then recovered by centrifugation 92. After recovered by the centrifugal separator, the eluted DNA sample is subjected to electrophoresis such that the precision of the selectivity of hybridization can be determined based on a band width after electrophoresis.

### EXAMPLE 3

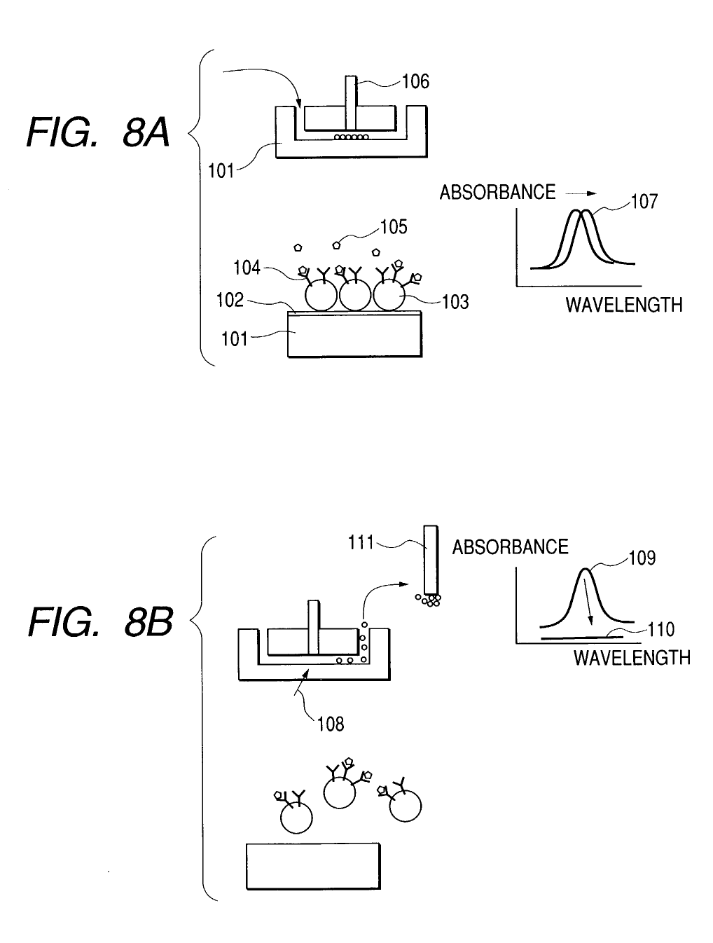

FIGS. 8A and 8B show another example according to the present invention. The inside of a fine channel 101 made of poly(methyl methacrylate) (PMMA) is coated with a gold thin film 102 with a thickness of 20 nm and a layer of magnetic beads 103 attached thereon. The magnetic beads 103 have a particle diameter of 30 nm to 300 nm and are made of dextran. A film of gold with a thickness of from 10 nm to 25 nm is formed on an upper half of each magnetic bead 103 by vapor deposition. This structure has a significant absorption spectrum in visible light regions, and its maximal absorption wavelength varies depending on the refractive index of the surface of the fine particle. When a growth factor receptor 104 is immobilized on the surface of the gold-deposited fine particle and a specimen containing a growth factor 105 is supplied into the fine channel 101, the binding between the growth factor 105 and the receptor 104 can be optically monitored. Specifically, the gold-deposited fine particle is irradiated with light in an optical fiber bundle 106, and the reflected light is introduced into a spectrophotometer through a coaxial optical fiber. The maximal absorption wavelength 107 of the reflection spectrum shifts before and after the attachment of the growth factor (FIG. 8A). After checking to ensure that the growth factor is attached, five pulses of double harmonic waves 108 of YAG at an intensity of 5 mJ/cm² are applied to the gold-deposited fine particle. At the time when the gold-deposited fine particle is separated from the substrate, absorbance at the maximal absorption wavelength shifts from equal to or more than 2 (109) to less than or equal to 0.5 (110) so as to optically monitor how the fine particle is separated from the substrate. The separated fine particle is extracted from the fine channel 101 and can easily be recovered using a magnet 111 (FIG. 8B). To verify its in-vivo activity, the growth factor is eluted and is added to cultured cells, such as nerve cells.

### EXAMPLE 4

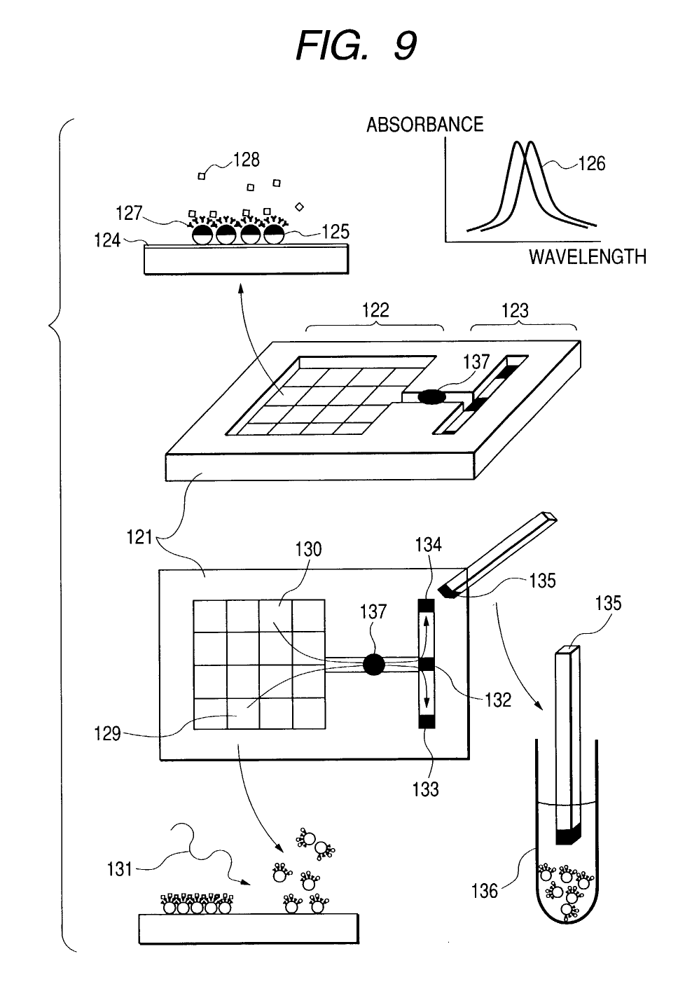

FIG. 9 shows another example according to the present invention. A chip 121 made of polystyrene includes a biomolecule attachment unit 122 and a biomolecule recovery unit 123. The biomolecule attachment unit 122 carries a gold thin film 124 formed on its bottom. A layer of magnet beads 125 made of dextran with a diameter of 30 nm to 300 nm are attached to the gold thin film 124. A film of gold with a thickness of 10 nm to 25 nm is formed on an upper half of each magnetic bead 125 by vapor deposition. This structure has a significant absorption spectrum 126 in visible light regions, and its maximal absorption wavelength varies depending on the refractive index of the surface of the fine particle. When a receptor 127 is immobilized onto the surface of the gold fine particle and a specimen containing a kind of target ligand 128 is added, the binding between the ligand 128 and the receptor 127 can be optically monitored. The biomolecule attachment unit 122 is divided into plural regions, and the magnet beads in different regions are modified or labeled with different receptors, respectively. Accordingly, this configuration can concurrently detect plural target ligands. The case in which target molecules are to be attached to a region A 129 and a region B 130 is taken as an example below. The sequence of attracting and releasing the magnetic beads 125 by the electromagnets is also given as an example.

Initially, one pulse of nitrogen laser light 131 at an intensity of 10 mJ/cm² is applied to the region A 129 to separate the magnetic beads 125 from the region A 129. The magnetic beads 125 are drawn using a pump 137, and concurrently, an electromagnet A 132 is energized to generate a magnetic field with a surface inductive flux of 1000 gausses to collect the magnetic beads 125 onto the electromagnet A 132.

Next, an electromagnet B 133 is energized to generate a magnetic field with a surface inductive reflux of 1000 gausses, and concurrently, the electromagnet A 132 is nonenergized to release the magnetic beads 125 onto the electromagnet B 133. The magnetic beads 125 are then treated with a buffer to elute the ligand, and the eluted ligand can be analyzed using a mass spectrometer.

Next, one pulse of nitrogen laser light 131 at an intensity of 10 mJ/cm² is applied to the region B 130 to separate the magnetic beads from the region B 130. The magnetic beads 125 are drawn using a pump 137, and concurrently, the electromagnet A 132 is energized to generate a magnetic field with a surface inductive flux of 1000 gausses to release the magnetic beads 125 onto the electromagnet A 132. Next, an electromagnet C 134 is energized to generate a magnetic field with a surface inductive reflux of 1000 gausses, and concurrently, the electromagnet A 132 is nonenergized to thereby release the magnetic beads 125 onto the electromagnet C 134. An electromagnet D 135 is placed in the vicinity of the electromagnet C 134 and is energized to generate a magnetic field with a surface inductive flux of 1000 gausses, and concurrently, the electromagnet C 134 is nonenergized to allow the electromagnet D 135 to attract the magnetic beads 125. After the attachment, the electromagnet D 135 is placed into a tube 136 and the electromagnet D 135 is nonenergized. Thus, the magnetic bead 125 can be transferred into the tube 136. By these procedures, the ligand captured on the surface of the magnetic beads 125 can be easily recovered and subjected to a subsequent analysis without loss.

### EXAMPLE 5

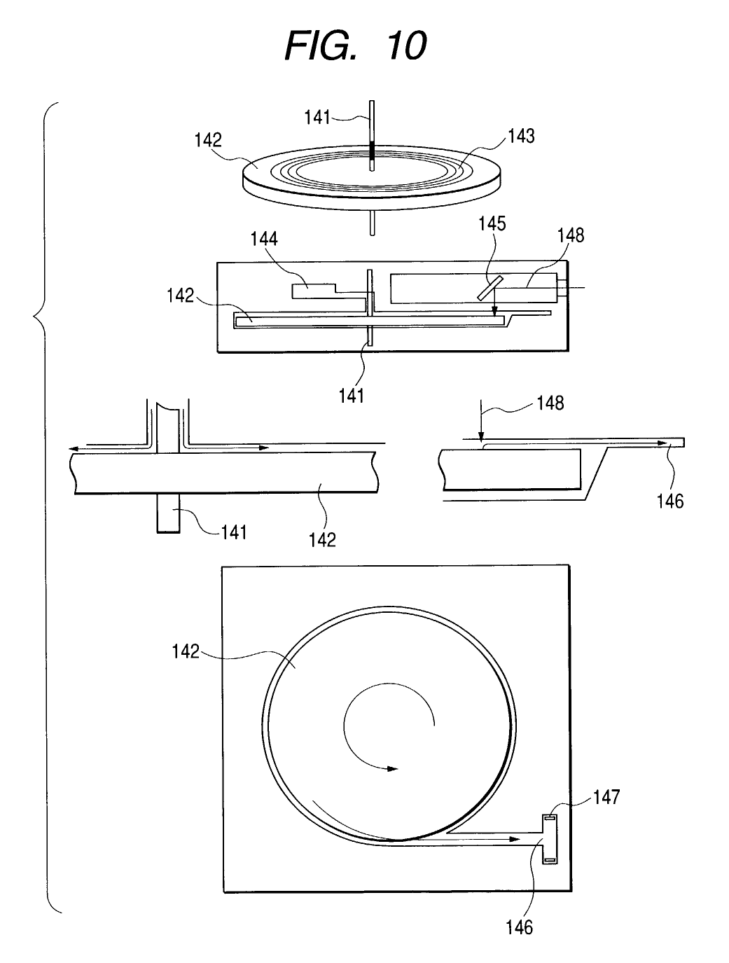

FIG. 10 shows another example according to the present invention. In this configuration, a disc 142 with a diameter of 1 cm to 30 cm is supported by a rotation axis 141. A layer of magnetic beads made of dextran and with a diameter of 100 nm are attached to the disc 142. The upper half of each magnetic bead is coated with a film of gold with a thickness of 20 nm. The fine particle layer on the disc 142 is divided into plural ring-shaped regions 143, and the magnetic beads in different regions are modified or labeled with different receptors. This configuration can concurrently detect plural target ligands. A specimen in a specimen chamber 144 is supplied from the vicinity of a rotation axis while rotating the disc 142 at a revolutions rate of 1 to 50 rpm. The specimen contains plural types of bacteriophages expressing peptides on their surfaces. Each of the bacteriophages is captured when the surface peptide is bound with the corresponding receptor. Next, the revolution rate of the disc 142 is increased to 10000 rpm, and one pulse of excimer laser light 144 at an intensity of 1 mJ/cm² is applied to separate the polystyrene fine particle from the disc 142. A region to be irradiated is controlled by the movement of a mirror 145. The separated beads are separated by a centrifugal force and is fed into a recovery channel 146. A pair of electrodes 147 are arranged at the back of the recovery channel 146, and a voltage of 2 V is applied between the electrodes 147. Thus, the target bacteriophages can be separated from the beads due to electric charge of the beads. By using the recovered bacteriophages, a peptide having an excellent specific binding capacity can be produced in large quantity.

### EXAMPLE 6

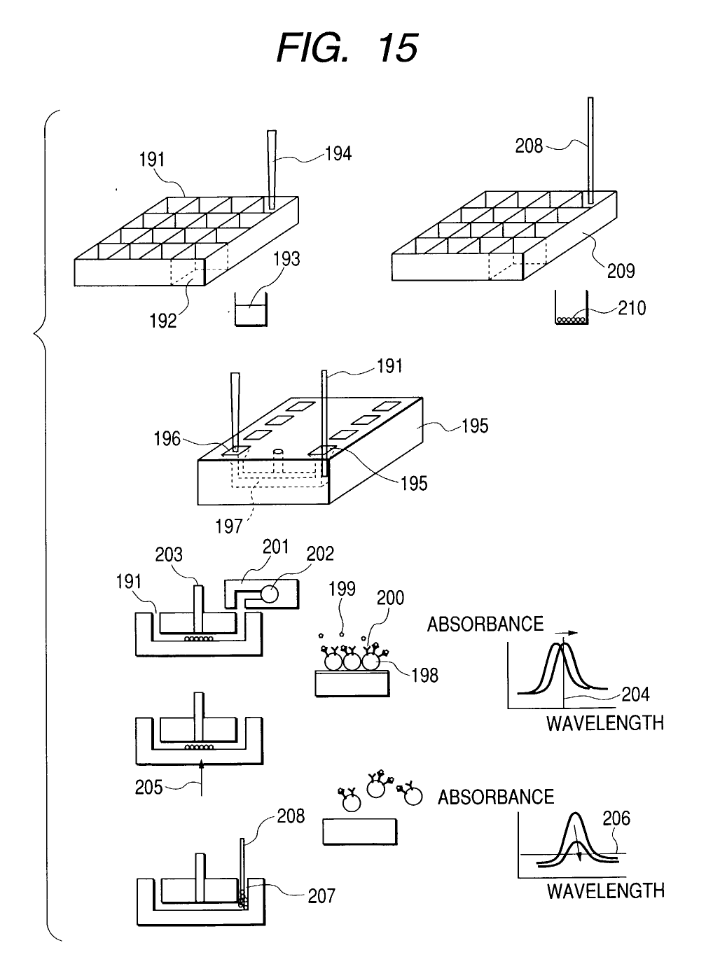

FIG. 15 shows another example according to the present invention. A chamber 191 is divided into plural wells 192, and each of the wells 192 contains an unpurified organic specimen 193. The unpurified organic specimen 193 is automatically purified according to the following process. Specifically, the specimen 193 is drawn from the well using a pipette 194 controlled by a robot. The pipette is moved to a purification block 195 and is injected into a fine channel 197 via a specimen inlet 196. The fine channel 197 carries, at its bottom, polystyrene fine particles 198 with a diameter of 100 nm and being coated with a film of gold with a thickness of 20 nm. The polystyrene fine particle 198 is modified or labeled with an antibody 200 capable of selectively binding with a target biomolecule 199 to be purified. A pump block 201 is locked on the purification block 195, and the unpurified specimen 193 is fed into the vicinity of the polystyrene fine particles 198 by action of a pump 202. The unpurified specimen 193 can be moved to and from the purification block 195 by periodically reversing the direction of movement of the pump 202. When the antibody 200 captures the target biomolecules 199 in the unpurified specimen 193, the optical characteristics of the polystyrene fine particles 198 vary. The maximal absorption wavelength of a absorption spectrum shifts to a longer wavelength side. The shift is monitored by an optical fiber bundle 203. The degree of the shift of the maximal absorption wavelength increases with an increasing amount of the captured target biomolecules 199. At the time when the maximal absorption wavelength reaches a predetermined wavelength 204, a cleaning fluid is injected into the fine channel 197 to wash the polystyrene fine particle 198 so as to remove impurities other than the target biomolecules. After washing, the pump 202 is stopped, and the pump block 201 is separated from the purification block 195. Next, a double harmonic wave 205 of YAG at an intensity of 1 mJ/cm² is applied to the fine channel 197 from its bottom. The polystyrene fine particle 198 is separated by laser irradiation, and the absorbance in an absorption spectrum decays with separation of the polystyrene fine particle 198. At the time when the absorbance becomes below a predetermined absorbance 206, it is determined that the overall polystyrene fine particles 198 have separated and the laser irradiation is stopped. Next, an electromagnet 208 is inserted from an outlet 207 of the purification block so as to generate a magnetic field with a surface inductive flux of 3000 gausses to collect the polystyrene fine particles 198 containing a super paramagnetic fine particle. The electromagnet 208 generating the magnetic field is moved into a well 210 in a chamber 209, and the electromagnet 208 is then nonenergized to release the polystyrene fine particles 198. The biomolecules are purified and concentrated with a minimal amount of a buffer 210 to avoid denaturation of the purified biomolecules and they are stored.

The present invention thus configured can efficiently recover specimens such as biomolecules almost without loss.

In summary, the present invention (1) captures and detects target biomolecules without use of any labeling (such as fluorescence, radio isotope); (2) releases the captured biomolecules with full knowledge of the amount of the released sample; (3) transfers under full control the biomolecules almost without loss; and (4) almost completely collects the transferred sample. In contrast, the prior art (1) captures and detects target biomolecules by labeling (such as fluorescence, radioisotope); (2) releases the captured biomolecules with limited knowledge of the amount of the released sample; (3) transfers the biomolecules with limited control and significant loss; and (4) collects the biomolecules under many restraints.

In addition, the present invention can skip detecting the biomolecules but go straight to capturing and recovering biomolecules. In the prior art reference involving magnetic beads, the use of a magnet to immobilize magnetic beads is routinely carried out. On the other hand, the present invention does not require magnetic beads for immobilization. Instead, plain beads (for cost concern) or beads with properties other than magnetic property are more suitable for the biomolecule recovery. Moreover, once the beads are adsorbed on a substrate according to the present invention, it does not require anything additionally to keep the beads immobilized. For example, the beads can be formed on a simple laminated piece of paper. Also, the present invention allows a wide range of removal methods, whereas the prior art inevitably requires the physical removal of the magnet or shutting down of the electromagnet. Finally, with magnetic beads, one hardly has control over the amount of beads on the surface, wherein the present invention, we quantitatively know exactly how many particles and thereby how man probe molecules are on the surface.

Other embodiments and variations will be obvious to those skilled in the art, and this invention is not to be limited to the specific matters stated above.

The principles, preferred embodiments and modes of operation of the present invention have been described in the foregoing specification. However, the invention which is intended to be protected is not limited to the particular embodiments disclosed. The embodiments described herein are illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. A method for recovering biomolecules, comprising:
bringing a specimen containing target biomolecules into contact with at least one fine particle being formed on a substrate for carrying at least one probe so as to bind the probe with the target biomolecules;
monitoring the biomolecules on the substrate by using an optical property of the fine particle without labeling the biomolecules;
separating and collecting the fine particle from the substrate by a physical property of the fine particle; and
recovering the biomolecules from the fine particle.

2. The method according to claim 1, wherein the fine particle is a noble metal-dielectric composite fine particle.

3. The method according to claim 1, further comprising a step of optically checking attachment of the biomolecule using the probe.

4. The method according to claim 3, wherein the optically checking step involves:
irradiating the biomolecules with a light so as to provide a reflected light, and
checking a change of a maximal absorption wavelength of the reflected light.

5. The method according to claim 1, wherein the separating step involves applying an ultrasonic wave to the substrate.

6. The method according to claim 1, wherein the separating step involves applying a pulsed laser light to the substrate.

7. The method according to claim 1, wherein the separating step involves separating the fine particle from the substrate with a scraper or a pressure-sensitive adhesive tape.

8. The method according to claim 1, wherein the collecting step involves collecting the separated fine particle by centrifugal separation.

9. The method according to claim 1, wherein the collecting step involves collecting the separated fine particle with a filter.

10. The method according to claim 1, wherein the collecting step involves:
collecting the separated fine particle with a magnet,
wherein the fine particle contains a super paramagnetic substance.

11. The method according to claim 1, wherein the collecting step involves:
electrostatically collecting the separated fine particle,
wherein the fine particle carries an electric charge.

12. A method for recovering biomolecules according to claim 1, further comprising a step of:
determining a recovery rate of the biomolecules.

13. The method according to claim 12, wherein the determining step involves determining a light absorption rate or a light scattering rate of the fine particle.

14. The method according to claim 12, wherein the determining step involves:
determining a fluorescence signal strength of a fluorescent dye contained in the fine particle.

15. An apparatus for recovering biomolecules, comprising:
a substrate carrying at least one fine particle on its surface, and the fine particle carries at least one probe;
an inlet for importing a specimen containing the biomolecules capable of being bound with the probe;
a measuring instrument for determining attachment of the biomolecules to the substrate by the probe;
a monitoring means for monitoring the biomolecules attached to the substrate using an optical property of the fine particle without labeling the biomolecules;
a separation unit for separating the fine particle from the substrate; and
a recovery unit for recovering the fine particle.

16. An apparatus for recovering biomolecules according to claim 15, wherein the substrate is divided into plural sections for accommodating plural kinds of target biomolecules.

17. The method according to claim 1, further comprising a step of dividing the substrate into plural sections for accommodating plural kinds of target biomolecules.

18. The method according to claim 13, wherein the determining step involves applying a near-infrared light to the find particle.

19. A method for recovering biomolecules, comprising:
bringing a specimen containing target biomolecules into contact with at least one noble metal-dielectric composite fine particle being formed on a substrate for carrying at least one probe so as to bind the biomolecules with the probe;
separating and collecting the fine particle from the substrate by a physical property of the fine particle; and
recovering the biomolecules from the find particle.

20. A method according to claim 19, further comprising a step of determining a recovery rate of the biomolecules.
